(19) United States
(12) Patent Application Publication
(10) Pub. No.: US 2024/0308949 A1
(43) Pub. Date: Sep. 19, 2024

(54) LIQUID-PHASE OXIDATION OF METHACROLEIN WITH GOLD BASED CATALYSTS

(71) Applicants: Dow Global Technologies LLC, Midland, MI (US); Rohm and Haas Company, Collegeville, PA (US)

(72) Inventors: Jing L. Houser, Midland, MI (US); Dmitry A. Krapchetov, Lansdale, PA (US)

(21) Appl. No.: 18/257,654

(22) PCT Filed: Dec. 2, 2021
(86) PCT No.: PCT/US2021/061513

§ 371 Date: Jun. 15, 2023

Related U.S. Application Data
(63) Continuation of application No. 63/127,643, filed on Dec. 18, 2020.

(51) Int. Cl. C07C 51/25; B01J 21/04; B01J 21/06; B01J 23/06; B01J 23/10; B01J 23/52; B01J 23/66

(52) U.S. Cl. CPC C07C 51/252; B01J 21/04; B01J 21/063; B01J 21/066; B01J 23/06; B01J 23/10; B01J 23/52; B01J 23/66

(57) ABSTRACT

A method for preparing methacrylic acid from methacrolein. The method comprises contacting a liquid mixture comprising methacrolein and water in the presence of oxygen with a heterogeneous catalyst comprising a support and gold; wherein the support comprises an oxide selected from γ-, δ-, or θ-alumina, magnesia, titania, zirconia, hafnia, vanadia, niobium oxide, tantalum oxide, ceria, yttria, lanthanum oxide, zinc oxide or a combination thereof.

### BACKGROUND OF THE INVENTION

The invention relates to a method for preparing methacrylic acid from liquid-phase oxidation of methacrolein using a gold based catalyst.

Acrylic acid, methacrylic acid, and their esters are important chemicals with widespread uses for the manufacture of various polymers, coatings, adhesives, elastomers, and other products.

Traditionally, methacrylic acid has been prepared from acetone cyanohydrin. Alternatively, methacrylic acid can be made by the gas-phase oxidation of isobutylene or tert-butanol to methacrolein, which is subsequently converted to methacrylic acid.

Liquid-phase oxidation is an attractive alternative to gas-phase oxidation because liquid-phase oxidation is expected to require milder conditions and smaller equipment.

Several attempts have been made to prepare methacrylic acid by liquid-phase oxidation. For example, U.S. Patent Application Publication No. 2015/0321178 discloses a process for producing carboxylic acids using a noble metal on a silica-based support comprising silicon, aluminum, and at least one element selected from iron, cobalt, nickel, and zinc.

There is a need for an improved process for the liquid-phase oxidation of methacrolein to produce methacrylic acid.

### SUMMARY OF THE INVENTION

The present invention is directed to a method for preparing methacrylic acid from methacrolein. The method comprises contacting a liquid mixture comprising methacrolein and water in the presence of oxygen with a heterogeneous catalyst comprising a support and gold, wherein the support comprises an oxide selected from γ-, δ-, or θ-alumina, magnesia, titania, zirconia, hafnia, vanadia, niobium oxide, tantalum oxide, ceria, yttria, lanthanum oxide, zinc oxide or a combination thereof.

### DETAILED DESCRIPTION OF THE INVENTION

The present invention is directed to the liquid-phase oxidation of methacrolein to form methacrylic acid.

In the method of the present invention, a liquid mixture comprising methacrolein and water is contacted with a heterogeneous catalyst in the presence of oxygen.

The heterogeneous catalyst comprises gold on a support. The support comprises a particle of an oxide selected from γ-, δ-, or θ-alumina, magnesia, titania, zirconia, hafnia, vanadia, niobium oxide, tantalum oxide, ceria, yttria, lanthanum oxide, zinc oxide or a combination thereof. Preferably, the support comprises γ-, δ-, or θ-alumina, titania, ceria, zinc oxide or a combination thereof. More preferably, the support comprises titania.

Preferably, the support does not comprise undoped silica or silicon carbide. For example, the support may comprise ceria doped silica, but may not comprise undoped silica.

Preferably, in portions of the catalyst comprising the noble metal, the support has a surface area greater than 10 m²/g, preferably greater than 30 m²/g, preferably greater than 50 m²/g, preferably greater than 100 m²/g, preferably greater than 120 m²/g. In portions of the catalyst which comprise little or no noble metal, the support may have a surface area less than 50 m²/g, preferably less than 20 m²/g.

Preferably, at least 90 wt % of the noble metal(s) is in the outer 50% of catalyst volume (i.e., the volume of an average catalyst particle), preferably the outer 40%, preferably in the outer 35%, preferably in the outer 30%, preferably in the outer 25%. Preferably, the outer volume of any particle shape is calculated for a volume having a constant distance from its inner surface to its outer surface (the surface of the particle), measured along a line perpendicular to the outer surface.

For example, for a spherical particle the outer x % of volume is a spherical shell whose outer surface is the surface of the particle and whose volume is x % of the volume of the entire sphere. Preferably, at least 95 wt % of the noble metal is in the outer volume of the catalyst, preferably at least 97 wt %, preferably at least 99 wt %. Preferably, at least 90 wt % (preferably at least 95 wt %, preferably at least 97 wt %, preferably at least 99 wt %) of the noble metal(s) is within a distance from the surface that is no more than 15% of the catalyst diameter, preferably no more than 10%, preferably no more than 8%, preferably no more than 6%. Distance from the surface is measured along a line which is perpendicular to the surface. The “catalyst center” is the centroid of the catalyst particle, i.e., the mean position of all points in all coordinate directions. A diameter is any linear dimension passing through the catalyst center and the average diameter is the arithmetic mean of all possible diameters.

The aspect ratio is the ratio of the longest to the shortest diameters. Preferably, the aspect ratio of the catalyst particle is no more than 10:1, preferably no more than 5:1, preferably no more than 3:1, preferably no more than 2:1, preferably no more than 1.5:1, preferably no more than 1.1:1. Preferred shapes for the catalyst particle include spheres, cylinders, rectangular solids, rings, multi-lobed shapes (e.g., cloverleaf cross section), shapes having multiple holes and “wagon wheels;” preferably spheres. Irregular shapes may also be used.

Preferably, the average diameter of the catalyst particle is at least 200 microns, preferably at least 400 microns, more preferably at least 600 microns, and even preferably at least 800 microns. The average diameter of the catalyst particles is preferably no more than 30 mm, more preferably no more than 20 mm, and even more preferably no more than 10 mm. The average diameter of the support and the average diameter of the final catalyst particle are not significantly different.

Preferably, the amount of gold as a percentage of the gold and the support is from 0.2 to 5 wt %, preferably at least 0.5 wt %, preferably at least 0.8 wt %, preferably at least 1 wt %, preferably at least 1.2 wt %; preferably no more than 4 wt %, preferably no more than 3 wt %, preferably no more than 2.5 wt %.

Preferably, the catalyst is produced by precipitating the gold from an aqueous solution of a gold salt in the presence of the support. In one embodiment of the invention, the catalyst is produced by incipient wetness in which an aqueous solution of a suitable gold precursor salt is added to a porous inorganic oxide such that the pores are filled with the solution and the water is then removed by drying. Preferred salts include tetrachloroauric acid, sodium aurothiosulfate, sodium aurothiomalate, and gold hydroxide. The resulting material is then converted into a finished catalyst by calcination, reduction, or other treatments known to those skilled in the art to decompose the gold salts into metals or metal oxides. Preferably, a C₂-C₁₈ thiol comprising at least one hydroxyl or carboxylic acid substituent is present in the solution. Preferably, the C₂-C₁₈ thiol comprising at least one hydroxyl or carboxylic acid substituent has from 2 to 12 carbon atoms, preferably 2 to 8, preferably 3 to 6. Preferably, the thiol compound comprises no more than 4 total hydroxyl and carboxylic acid groups, preferably no more than 3, preferably no more than 2. Preferably, the thiol compound has no more than 2 thiol groups, preferably no more than one. If the thiol compound comprises carboxylic acid substituents, they may be present in the acid form, conjugate base form or a mixture thereof. The thiol component also may be present either in its thiol (acid) form or its conjugate base (thiolate) form. Especially preferred thiol compounds include thiomalic acid, 3-mercaptopropionic acid, thioglycolic acid, 2-mercaptoethanol and 1-thioglycerol, including their conjugate bases.

In one embodiment of the invention, the catalyst is produced by deposition precipitation in which a porous inorganic oxide is immersed in an aqueous solution containing a suitable gold precursor salt and that salt is then made to interact with the surface of the inorganic oxide by adjusting the pH of the solution. The resulting treated solid is then recovered (e.g. by filtration) and then converted into a finished catalyst by calcination, reduction, or other treatments known to those skilled in the art to decompose the gold salts into metals or metal oxides.

The catalyst particles are preferably contained in a catalyst bed typically are held in place by solid walls and by screens or catalyst support grids. In some configurations, the screens or grids are on opposite ends of the catalyst bed and the solid walls are on the side(s), although in some configurations the catalyst bed may be enclosed entirely by screens. Preferred shapes for the catalyst bed include a cylinder, a rectangular solid and a cylindrical shell; preferably a cylinder.

The liquid mixture comprises methacrolein and water. The water may be present in the liquid mixture in an amount ranging from 1 to 95 wt % relative to the total weight of the liquid mixture. Preferably, the water is present in an amount of at least 5 wt %, more preferably at least 10 wt %, even more preferably at least 15 wt %, and still more preferably at least 20 wt % relative to the total weight of the liquid mixture. Preferably, the water is present in an amount less than 90 wt % relative to the total weight of the liquid mixture.

The methacrolein may be present in the liquid mixture in an amount ranging from 2 to 60 wt % relative to the total weight of the liquid mixture. Preferably, the methacrolein is present in an amount of at least 3 wt %, more preferably at least 5 wt %, and even more preferably at least 10 wt % relative to the total weight of the liquid mixture. Preferably, the methacrolein is present in an amount less than 50 wt % relative to the total weight of the liquid mixture, such as for example, less than 40 wt % or less than 30 wt %.

The liquid mixture may further comprise an organic solvent. Preferably, the organic solvent has an oxygen solubility greater than that of water (8.2 ppm at 25° C. and 1 atm (101 kPa)). For example, the organic solvent may have an oxygen solubility greater than 100 ppm at 25° C. and 1 atm (101 kPa). More preferably, the organic solvent has an oxygen solubility greater than 150 ppm at 25° C. and 1 atm (101 kPa), and even more preferably greater than 300 ppm at 25° C. and 1 atm (101 kPa). Without wishing to be bound by theory, it is believed that the addition of an organic solvent with an oxygen solubility higher than that of water may help increase the capability of the liquid mixture to supply oxygen for the reaction.

Examples of the organic solvents that may be present in the liquid mixture include, but are not limited to acetonitrile, toluene, o-xylene, hexane, octane, N,N-dimethyl formamide, Fluorinert™ FC-770, dichloroethane, acetone, and diglyme (bis(2-methoxyethyl) ether). Preferably, the organic solvent comprises acetone, N,N-dimethyl formamide, or diglyme. Even more preferably, the organic solvent comprises diglyme.

The organic solvent may be miscible with water. As defined herein, an organic solvent is miscible with water if a mixture forms a homogeneous solution.

In the method of the present invention, the liquid mixture is contacted with the heterogeneous catalyst in the presence of oxygen. The oxygen may be supplied to the reaction using air or oxygen, with or without an inert gas.

The reaction may take place, for example, at a temperature ranging from 40 to 120° C. Preferably, the temperature is at least 50° C., and more preferably at least 60° C. The temperature is preferably less than 110° C., and more preferably less than 100° C.

The reaction may take place at a pressure ranging from 0.5 to 100 bar; preferably no more than 80 bar, and more preferably no more than 20 bar.

### EXAMPLES

All examples were run with a 300 ml Parr reactor. All the reactions were run at 80° C. and 100 psig (790 kPa). The gas feed consisted of 8% of O₂ and 92% of N₂. The limited O₂ concentration in the gas feed was to ensure the avoidance of the reactants' and the solvents' flammable zones. 200-500 ppm of hydroquinone (HQ) was used in each run as polymerization inhibitor. A series of solvents was used for the reaction. 3 g of gold based catalysts with different supports were used for catalyzing the oxidative reaction. The reaction time was 1 hr and 20 min for most of the runs, except that two runs lasted for 2 hr. The final reaction mixtures were analyzed with gas chromatography (GC).

### EFFECT OF WATER CONCENTRATION

Experiments showing the impact of water on the liquid phase oxidation of methacrolein (MA) to methacrylic acid (MAA) with various levels of acetone solvent are listed in Table 1. As shown in Table 1, the presence of water greatly affected the reaction, while the presence of acetone did not affect the reaction as much. As the concentration of water increased from zero to 90%, both MAA selectivity and space time yield increased. MAA selectivity eventually surpassed 90% when the water level was 25% or higher; the space time yield appeared to exhibit a maximum.

TABLE 1

| | | | | | | | MAA space |
|---|---|---|---|---|---|---|---|
| | | | | | | | time yield |
| | | Reaction | | wt % | wt % | MAA | (mol/kg- |
| | Catalyst | time (hr) | Solvent | water | MA | selectivity | cat/hr) |
| Ex. 1 | Au/TiO2 | 1.33 | none | 90.0% | 10.0% | 98.30% | 3.11 |
| Ex. 2 | Au/TiO2 | 1.33 | acetone | 58.0% | 10.0% | 92.67% | 5.27 |
| Ex. 3 | Au/TiO2 | 1.33 | acetone | 25.0% | 10.0% | 95.58% | 4.98 |
| Ex. 4 | Au/TiO2 | 1.33 | acetone | 2.5% | 10.0% | 87.98% | 1.47 |
| Comp. | Au/TiO2 | 1.33 | acetone | 0.0% | 10.0% | 0.00% | 0 |
| Ex. 1 | | | | | | | |

### EFFECT OF SOLVENT

Table 2 shows the experiments with various solvents at similar experimental conditions (i.e., temperature, pressure, reaction time, the amount of catalyst used, etc.) According to the results shown in Table 2, high selectivity was achieved in N,N-dimethylformamide, acetone and diglyme, as well as without solvent, with diglyme also exhibiting the highest activity. For some of the reactions whose solvents had significant O₂ solubility, no water was added. These examples confirmed that MAA was not produced in measurable amounts in the absence of water.

TABLE 2

| | | | | | | | MAA space |
|---|---|---|---|---|---|---|---|
| | | Reaction | | | | | time yield |
| | | time | | wt % | wt % | MAA | (mol/kg- |
| | Catalyst | (h) | Solvent | water | MA | selectivity | cat/hr) |
| Ex. 5 | Au/TiO2 | 2 | acetonitrile | 63.0% | 5.0% | 97.62% | 0.02 |
| Comp. | Au/TiO2 | 2 | toluene | 0.0% | 6.7% | Almost no MAA | |
| Ex. 2 | | | | | | detected by GC. | |
| Comp. | Au/TiO2 | 1.33 | o-xylene | 0.0% | 10.0% | Almost no MAA | |
| Ex. 3 | | | | | | detected by GC. | |
| Comp. | Au/TiO2 | 1.33 | hexane | 0.0% | 5.0% | Almost no MAA | |
| Ex. 4 | | | | | | detected by GC. | |
| Comp. | Au/TiO2 | 1.33 | octane | 0.0% | 6.7% | Almost no MAA | |
| Ex. 5 | | | | | | detected by GC. | |
| Ex. 6 | Au/TiO2 | 1.33 | N,N-dimethyl | 50.0% | 6.7% | 97.54% | 4.53 |
| | | | formamide | | | | |
| Comp. | Au/TiO2 | 1.33 | Fluorinert ™ | 0.0% | 10.0% | Almost no MAA | |
| Ex. 6 | | | FC-770 | | | detected by GC. | |
| Comp. | Au/TiO2 | 1.33 | dichloroethane | 0.0% | 6.7% | Very little MAA | |
| Ex. 7 | | | | | | detected by GC. | |
| Ex. 7 | Au/TiO2 | 1.33 | acetone | 58.0% | 10.0% | 92.67% | 5.27 |
| Ex. 8 | Au/TiO2 | 1.33 | diglyme | 50.0% | 5.0% | 97.07% | 9.59 |
| Ex. 9 | Au/TiO2 | 1.33 | none | 90.0% | 10.0% | 98.30% | 3.11 |

### EFFECT OF CATALYST SUPPORT

Table 3 compares the performance of various catalyst supports. Most catalysts exhibited 90-95+% MAA selectivity. In terms of activity, the ranking of the space time yield of MAA is: Au/TiO₂>Au/Al₂O₃, Au/Ti-Q10, Au/CeO₂ doped SiO₂>other catalysts.

TABLE 3

| | | | | | | | MAA space |
|---|---|---|---|---|---|---|---|
| | | Reaction | | | | | time yield |
| | | time | | wt % | wt % | MAA | (mol/kg- |
| | Catalyst | (h) | Solvent | water | MA | selectivity | cat/hr) |
| Ex. 10 | Au/TiO2 | 2 | Acetonitrile | 63.0% | 5.0% | 97.62% | 0.02 |
| Comp. | Au/SiO2 | 1.33 | Acetonitrile | 63.0% | 5.0% | Almost no MAA | |
| Ex. 8 | | | | | | detected by GC. | |
| Ex. 11 | Au/TiO2 | 1.33 | N,N-dimethyl | 50.0% | 6.7% | 97.54% | 4.53 |
| | | | formamide | | | | |
| Comp. | Au/SiO2 | 1.33 | N,N-dimethyl | 50.0% | 6.7% | Almost no MAA | |
| Ex. 9 | | | formamide | | | detected by GC. | |
| Ex. 12 | Au/TiO2 | 1.33 | acetone | 58.0% | 10.0% | 92.67% | 5.27 |
| Comp. | Au/SiO2 | 1.33 | acetone | 58.0% | 10.0% | Almost no MAA | |
| Ex. 10 | | | | | | detected by GC. | |
| Ex. 13 | Au/Al2O3 | 1.33 | acetone | 58.0% | 10.0% | 95.34% | 2.09 |
| Ex. 14 | Au/ZnO | 1.33 | acetone | 58.0% | 10.0% | 93.45% | 1.04 |
| Ex. 15 | Lab-scale | 1.33 | acetone | 58.0% | 10.0% | 94.67% | 2.25 |
| | Au/Ti-Q10 | | | | | | |
| Ex. 16 | Au/CeO2 | 1.33 | acetone | 58.0% | 10.0% | 93.28% | 2.69 |
| | Doped SiO2 | | | | | | |
| Ex. 17 | Au/ZrO2 | 1.33 | acetone | 58.0% | 10.0% | 94.47% | 1.79 |
| Comp. | Au/SiC | 1.33 | acetone | 58.0% | 10.0% | Almost no MAA | |
| Ex. 11 | | | | | | detected by GC. | |
| Ex. 18 | Pilot-scale | 1.33 | acetone | 58.0% | 10.0% | 96.37% | 2.79 |
| | Au/Ti-Q10 | | | | | | |
| Comp. | TIO2 | 1.33 | acetone | 58.0% | 10.0% | No MAA | |
| Ex. 12 | | | | | | detected by GC. | |
| Ex. 19 | Au/TiO2 | 1.33 | diglyme | 50.0% | 5.0% | 97.07% | 9.59 |
| Ex. 20 | Au/SiO2 | 1.33 | diglyme | 50.0% | 5.0% | 16.60% | 0.62 |

Unless otherwise indicated by the context of the specification, all amounts, ratios and percentages are by weight, and all test methods are current as of the filing date of this disclosure. The articles “a”, “an” and “the” each refer to one or more. It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, different, special, and/or unexpected results may be obtained from each member of the respective Markush group independent from all other Markush members. Each member of a Markush group may be relied upon individually and or in combination and provides adequate support for specific embodiments within the scope of the appended claims.

Further, any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The term “composition,” as used herein, includes material(s) which comprise the composition, as well as reaction products and decomposition products formed from the materials of the composition.

The term “comprising,” and derivatives thereof, is not intended to exclude the presence of any additional component, step or procedure, whether or not the same is disclosed herein. In order to avoid any doubt, all compositions claimed herein through use of the term “comprising” may include any additional additive, adjuvant, or compound, whether polymeric or otherwise, unless stated to the contrary. In contrast, the term, “consisting essentially of” excludes from the scope of any succeeding recitation any other component, step or procedure, excepting those that are not essential to operability. The term “consisting of” excludes any component, step or procedure not specifically delineated or listed.

1. A method for preparing methacrylic acid from methacrolein comprising:
contacting a liquid mixture comprising methacrolein and water in the presence of oxygen with a heterogeneous catalyst comprising a support and gold; wherein the support comprises an oxide selected from γ-, δ-, or θ-alumina, magnesia, titania, zirconia, hafnia, vanadia, niobium oxide, tantalum oxide, ceria, yttria, lanthanum oxide, zinc oxide or a combination thereof.

2. The method of claim 1, wherein the water is present in the liquid mixture in an amount ranging from 1 to 95 wt % relative to the total weight of the liquid mixture.

3. The method of claim 2, wherein the water is present in the liquid mixture in an amount ranging from 20 to 90 wt % relative to the total weight of the liquid mixture.

4. The method of claim 1, wherein the liquid mixture further comprises an organic solvent.

5. The method of claim 4, wherein the organic solvent has an oxygen solubility greater than 8.2 ppm at 25° C. and 1 atm.

6. The method of claim 4, wherein the organic solvent is selected from the group consisting of N,N-dimethylformamide, acetone, and diglyme.

7. The method of claim 6, wherein the organic solvent comprises diglyme.

8. The method of claim 4, wherein the organic solvent is miscible in water.

9. The method of claim 1, wherein the catalyst has an average diameter from 200 microns to 10 mm.

10. The method of claim 1, wherein at least 90 wt % of the noble metal is in the outer 50% of catalyst volume.

11. The method of claim 10, wherein at least 95 wt % of the noble metal is in the outer 30% of catalyst volume.

12. The method of claim 1, wherein the support is selected from the group consisting of γ-, δ-, or θ-alumina, titania, ceria, and zinc oxide.

13. The method of claim 1, wherein the support comprises titania.

14. The method of claim 1, wherein the support does not comprise undoped silica or silicon carbide.
